(19) United States
(12) Patent Application Publication
(10) Pub. No.: US 2026/0078092 A1
(43) Pub. Date: Mar. 19, 2026

(54) SULFONYL CARBOXAMIDE COMPOUND, THEIR DERIVATIVES, AND USES THEREOF

(71) Applicant: Salim SHAH, Vienna, VA (US)

(72) Inventor: Salim SHAH, Vienna, VA (US)

(21) Appl. No.: 18/889,443

(22) Filed: Sep. 19, 2024

(51) Int. Cl. C07D 213/74; A61K 31/44; A61K 31/4427; A61K 31/4439; A61K 31/444; A61K 31/4545; C07D 401/12; C07D 471/08; C07D 487/08

(52) U.S. Cl. CPC C07D 213/74; A61K 31/44; A61K 31/4427; A61K 31/4439; A61K 31/444; A61K 31/4545; C07D 401/12; C07D 471/08; C07D 487/08

(57) ABSTRACT

The present invention relates to sulfonyl carboxamide derivatives represented by formula (I):

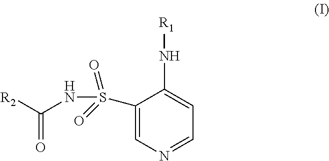

wherein R₁ represents an aryl group, a cycloalkyl group, a heterocycloalkyl group, or polycyclic group; and R₂ represents a bicyclic group. The invention further relates to a pharmaceutical composition comprising the compound of formula (I) in combination with one or more pharmaceutically acceptable excipients. This compound or composition specifically targets ion transporters, ensuring their proper functioning and preventing disorders associated with abnormal ion transport.

### FIELD OF THE INVENTION

The present invention relates to a sulfonyl carboxamide compound and pharmaceutical-acceptable salts thereof and their method of preparation for the treatment of diseases related to ion transports across the cell membrane.

### BACKGROUND OF THE INVENTION

Ion transport across the cell membrane is a dynamic process essential for maintaining cellular function and the overall physiological homeostasis of an organism. The cell membrane, also known as the plasma membrane, serves as a barrier between the intracellular and extracellular environments, maintaining the cells internal milieu and regulating the passage of ions and molecules, such as sodium (Na⁺), potassium (K⁺), calcium (Ca²⁺), and chloride (Cl⁻), which play pivotal roles in cellular processes including maintaining membrane potential, signal transduction, muscle contraction, nerve impulse propagation, and enzyme activation.

The movement of ions across the cell membrane occurs through various mechanisms including passive diffusion, facilitated diffusion, and active transport. Active transport is facilitated by various co-transporters that depend on an electrochemical gradient of ions across the cell's membrane. The co-transporters are integral membrane proteins that facilitate the simultaneous transport of two or more different ions or molecules across the cell membrane in the same direction. These transporters utilize the energy stored in the electrochemical gradient of one of the transported species to drive the movement of another against its concentration gradient. Co-transporters are crucial to maintaining various physiological processes, including nutrient absorption, ion homeostasis, and cellular signaling.

Chloride (Cl⁻) is a principal anion in the body, accounting for 70% of the total negative ion content and is transported by chloride (Cl⁻) channels, which are ubiquitously expressed across various mammalian tissues and cell types, and they perform a multiplicity of functions such as regulation of muscle contraction and membrane excitability, acidification of intracellular organelles, regulation of cell volume, trans-epithelial fluid transport, and modulation of the extracellular matrix.

Chloride (Cl⁻) channels at the cell membrane can be broadly categorized into the following classes based on their regulation mechanisms: i) calcium-activated Cl⁻ channels (CaCCs) or TMEM16 family; ii) maxi-Cl⁻ channel, iii) the CAMP-activated Cl⁻ channel CFTR (cystic fibrosis transmembrane conductance regulator); iv) CLC family or voltage-gated Cl⁻ channels; v) bestrophins; and vi) ligand-gated Cl⁻ channels (e.g. GABA, and glycine). Chloride (Cl⁻) channels are extensively expressed in the lung, and their impaired function or regulation contributes to the development of pulmonary diseases in both the lung parenchyma and the pulmonary blood vessels (Madhushri Sinha, Diana Zabini, Divya Guntur, Chandran Nagaraj, Peter Enyedi, Horst Olschewski, Wolfgang M. Kuebler, Andrea Olschewski, Chloride channels in the lung: Challenges and perspectives for viral infections, pulmonary arterial hypertension, and cystic fibrosis, Pharmacology & Therapeutics, Volume 237, 2022).

The intracellular chloride concentration (Cl⁻) is mostly controlled by a chloride-cation co-transporter (CCCs) of SLC12 gene family (Maa et al., 2011). The chloride-cation co-transporters are a group of membrane proteins that facilitate the coupled transport of chloride ions (Cl⁻) with cations such as sodium (Na⁺), potassium (K⁺), or calcium (Ca²⁺). These co-transporters are among the most important ion transporters in multicellular organisms, which are crucial for the survival of the organism (Alessi et al., 2014). Some of the common types of chloride-cation co-transporters (CCCs) include Na⁺—K⁺-2Cl⁻ co-transporter (NKCC), which mediate an electro-neutral uptake of one sodium ion, one potassium ion, and two chloride ions into the cell.

There are seven chloride co-transporters: one sodium chloride co-transporter (NCC), two sodium potassium chloride co-transporters (NKCC), and four potassium chloride transporters (KCC). Only one isoform of NCC is known at this time. There are two isoforms of NKCC: NKCC1, which is widely expressed in various tissues and cell types, and NKCC2, which is primarily found in the kidneys, particularly in the thick ascending limb of the loop of Henle. There are four isoforms of KCC: KCC1, KCC2, KCC3, and KCC4, each with distinct tissue distributions and physiological functions. The KCCs facilitate the electro-neutral transport of potassium and chloride ions across the cell membrane and are outwardly directed (Munoz, DeFelipe, & Alvarez-Leefmans, 2007). These transporters are targets of several approved drugs, such as diuretics (e.g., thiazide diuretics against NCC and loop diuretics against NKCC2).

The CCCs are intrinsic membrane proteins that use the energetically favourable transmembrane gradients of potassium and sodium ions to transport Cl⁻ ions across the cell membrane. The transmembrane gradients are established by an active transport of Na⁺K⁺-ATPase (Alessi et al., 2014). Thus, the Cl⁻ transport is performed electro-neutrally without any net charge movement across the cell membrane (Payne et al., 2003). NKCC1 and NKCC2 use the inward sodium current to transport Cl⁻ into the cell above its equilibrium level. The KCC1, KCC2, KCC3, and KCC4 use the potassium gradient to transport Cl⁻ out of the cell, lowering the Cl⁻ concentration below the equilibrium level (Maa et al., 2011). NKCCI is widely distributed throughout the body and expressed in neurons, glial cells, the choroid plexus, and vascular endothelium, whereas NKCC2 is primarily expressed in the kidneys (Maa et al., 2011). The NKCCI and NKCC2 share 60% homology at the protein level (Markadieu, N., Delpire, E., 2014).

The NKCC1 is highly expressed in the spiral and vestibular ganglia of an inner ear and regulates the Cl⁻ ions in adult neurons and is not located in the central nervous system (CNS). However, in the immature brain (during early development), there is an increased expression of NKCCI that elevates the level of Cl⁻ ions, contributing to the developmental effects during the early development stage (Dzhala et al., 2005). Further, NKCCI is also expressed in the salivary glands, and participates in the secretion of fluid and mucine. It is also expressed in the intestine, where it is involved in fluid excretion. The most striking deficit of NKCC1 knockout mice is deafness and imbalance, which originates from the fact that NKCC1 is highly expressed in the inner ear. It plays a major role in afferent neurons. In the CNS it is only elevated in immature neurons and plays an important role in neuronal maturation. NKCC1 knockout mice also suffer from hypotension and male infertility. The hypotension originates from a decreased vascular tone (Markadieu, N., Delpire, E., 2014).

The NKCC2 is expressed on the apical membrane of the epithelial cells in the ascending limb of the loop of Henle, which reabsorbs around 20-30% of the NaCl filtered by the glomerulus (Ares G., Caceres P., Ortiz P., 2011). The main function of the ascending limb of the loop of Henle is to reabsorb NaCl, without water, which leads to dilution of the forming urine in the tubular lumen. In addition, the NKCC2 is also expressed in macula densa, as the cells of the macula densa act as NaCl sensors to adjust the glomerular filtration by either vasoconstriction or vasodilation of afferent arteriole. The reabsorption of the NaCl in the loop of Henle causes the decrease of tubular NaCl concentration that leads to a vasodilation of the afferent arteriole and a release of renin by the granular cells. In contrast, if the concentration of the tubular NaCl increases it leads to a vasoconstriction of the afferent arteriole, thereby decreasing the glomerular filtration. This mechanism is known as the tubuloglomerular feedback where NKCC2 plays a pivotal role in sensing high levels of NaCl concentration (Peti-Peterdi, J., Harris, R., 2010).

In the mammalian central nervous system (CNS), the intracellular Cl⁻ ion concentration determines the strength and direction of GABAergic neurotransmission (Kahle & Staley, 2008). Since the adult CNS contains very low levels of Cl⁻ ion concentration, the activation of the γ-Aminobutyric acid receptor (GABA_{A}) leads to an influx of Cl⁻ into the cell, causing hyperpolarization and inhibition of neurons (Khanna, Walcott, & Kahle, 2013). The immature brain of neonates, on the other hand, exhibits a much higher Cl⁻, so that activating the GABA_{A} receptor causes an efflux of Cl⁻ ions, which depolarizes the neuron and leads to synaptic excitation of the neurons (Kahle & Staley, 2008).

The GABA_{A} receptor is a ligand-gated chloride channel opened by gamma-aminobutyric acid (GABA) docking to its binding site. GABA is a primary inhibitory neurotransmitter in the adult mammalian brain (Dzhala et al., 2005). GABA-mediated signaling also plays a key role in all important developmental steps, such as cell proliferation (Owens & Kriegstein, 2002). The GABA_{A} receptor, on reacting with GABA, undergoes a conformational change, which allows Cl⁻ ions to passively flow either into the cell or out of the cell, depending on the chloride equilibrium concentration (Maa et al., 2011). The ligand-gated chloride channel also allows bicarbonate to permeate the channel pore, but less efficiently than chloride (Owens & Kriegstein, 2002). The inflow of Cl⁻ ions causes hyperpolarization, whereas the outflow of Cl⁻ ions results in depolarization. In the immature brain, GABA has a depolarizing effect; it excites neurons and can therefore cause seizures (Dzhala et al., 2005). The depolarizing effect of GABA is crucial for brain development.

In the immature neuron, there is a delicate equilibrium between inhibition and excitation and this balance plays an important role during the early stages of brain development. Moreover, excessive inhibition leads to failure in neuronal growth and synaptic maturation, whereas excessive excitation can cause seizures and even excitotoxic death (Maa et al., 2011). In addition, the GABA_{A} receptor also influences DNA synthesis, proliferation, and neuronal migration (Owens & Kriegstein, 2002).

The disruptions in ion transport can lead to various physiological disorders. For example, malfunctioning of ion channels can cause channelopathies, which are characterized by abnormal electrical activity in cells and tissues. Cystic fibrosis, for instance, results from mutations in the cystic fibrosis transmembrane conductance regulator (CFTR) gene, leading to defective chloride ion transport across epithelial membranes. The disruption of ion channels further leads to hypertension, hyperkalemia, hypokalemia, heart failure, kidney disease, gout, anxiety autism spectrum, hyperhidrosis disorders, spinal cord injury, stroke, schizophrenia, edema, and Down syndrome etc.

Anxiety disorder affects around 18% of adults and is the most prevalent class of psychiatric disorders (Le'pine, J., 2002). The current treatment of anxiety disorders includes psychotherapy and medication, such as serotonin reuptake inhibitors (SSRIs), antidepressants, monoamine oxidase inhibitors, benzodiazepines, and anticonvulsants. However, 20-40% of the patients do not respond to any of these medications (Denys D., de Geus F. 2005). Moreover, it was shown that the drug bumetanide has an anxiolytic effect in rat models of conditioned anxiety. A rat treated with the drug bumetanide expressed a significantly smaller percentage of test periods freezing in a model of contextual fear conditioning than with the vehicle alone. The bumetanide also had a significant effect in the model of fear-potentiated startle (Krystal A D, Sutherland J, and Hochman D W 2012). Apart from no effect or response of the mentioned treatment methods on 20-40% of the patients having anxiolytic disorders, another downside of these drugs includes severe side effects in the CNS. Further, the long-term use of SSRIs can cause sexual dysfunction and weight gain (Hirschfeld, R., 2003).

Autism Spectrum Disorder (ASD) is a range of complex neuro-developmental disorders that are characterized by repetitive and characteristic patterns of behaviour and difficulties in social interaction and communication. The ASD consists of a range of different disorders, with autism being the most severe form. Other forms are Asperger Syndrome, childhood disintegrative disorder, and pervasive developmental disorders as part of ASD.

One out of every 68 children is affected by ASD and boys are more prone to develop ASD than girls. ASD occurs throughout all racial and ethnic groups and across all socioeconomic levels (NNDS Autism Spectrum Disorder Fact Sheet 2016). The drug bumetanide has also been used in an open-label trial with seven patients diagnosed with autism. The patients were treated with bumetanide for 10 months and with bumetanide use showing an improvement in emotion recognition and enhanced the activation of brain regions involved in social and emotional perception (Hadjikhani N., et al. 2015). This drug comes with certain drawbacks, notably the absence of a permanent cure and its limited effectiveness in treatment.

Hyperhidrosis is a medical condition that causes excessive sweating. It is classified as primary and secondary hyperhidrosis. Primary hyperhidrosis is idiopathic characterized by overactivity of the sympathetic nerves. It only afflicts a limited body area, mostly underarms, palms, soles, or head. While most of the body stays dry, one or two areas drip with sweat. Secondary hyperhidrosis results from side effects of certain medications or an underlying medical condition, such as diabetes or gout (Website of American Academy of Dermatology (2016)). Hyperhidrosis can have a substantial impact on everyday life, such as limitations in work, social interaction, physical activities, and mental and emotional health.

Available treatments for hyperhidrosis include topical aluminum chloride hexahydrate, oral anticholinergics, injectable botulinum toxin, or surgery. They all vary in their efficacy, side effects, cost, and ease of use. In the US approximately 15.3 million people are affected by hyperhidrosis, which accounts for approximately 4.8% of the total population. Out of these, only 51% of the affected people discussed their hyperhidrosis condition with a healthcare professional. 75% of the surveyed people report that their condition has had some negative impact on their social life, wellbeing, emotional or mental health. A large part of the population agrees that excessive sweating is embarrassing and leads to anxiety (Doolittle J., et al. 2016). The treatment of hyperhidrosis can be achieved by blocking the NKCC in sweat glands, which prevents excessive sweating. Further, the drug bumetanide is partly effective in treating hyperhidrosis.

Spinal cord injury (SCI) is another disorder that affects around 250,000 to 500,000 people worldwide each year. Around 90% of SCI cases are traumatic causes such as road traffic accidents, falls, or violence. The people affected by SCI have a two to five times increased risk of premature death. Symptoms of SCI depend on the severity of the injury and its location, but most patients experience chronic pain (WHO (2013) Spinal Cord Injury Fact Sheet N°384). Injury and noxious input can lead to a long-lasting increase in spinal cord neural excitability that may cause chronic pain. Spinal cord injury (SCI) can transform the activation of GABA channels from hyperpolarizing to depolarizing by down-regulating KCC2 leading to a high Cl⁻ concentration causing the shift of GABAergic activation. Neural injury pushes the spinal systems towards a state of early development, where GABA has a depolarizing effect. This depolarizing effect causes the development of chronic pain and spasticity.

The drug bumetanide can restore the normal functioning of GABAergic by blocking the NKCC and restoring normal Cl⁻ concentration (Huang Y., et al. 2016). This drug comes with certain drawbacks, notably the absence of a permanent cure and its limited effectiveness in treatment.

Stroke is the fifth leading cause of death in the US, which accounts for approximately 795,000 stroke cases in the US population each year. Strokes are caused by blocking or rupturing a blood vessel supplying blood to the brain. Stroke is also a major cause of disability and reduces mobility in more than half of stroke survivors older than the age of 65 years (Centers for Disease Control and Prevention, (2016) Stroke Fact Sheet).

Strokes are of various types including an ischemic stroke, which promotes adult neurogenesis, but these new neurons have rather limited capabilities to survive in the long term. Ischemic stroke can cause an imbalance in the expression of NKCC and KCC, leading to an increased Cl⁻ ions and ultimately to a shift in GABAergic activation from hyperpolarizing to depolarizing. Chronic post-treatment with the drug bumetanide can enhance the migration of neuroblasts toward the damaged striatum and can also enhance the survival of these newborn neurons. Furthermore, the behavioural assessment showed improved in beam-walking performance. Therefore, the drug bumetanide and its derivatives might cause a favourable microenvironment for newborn neurons that enhances their generation and survival (Xu W., et al. 2016). Thus, the bumetanide derivative can be used to enhance regeneration and to reduce damage after ischemic stroke, and also in other diseases (e.g., Alzheimer's disease) for memory enhancement.

Schizophrenia is another disorder characterized by distortions in emotions, perceptions, thinking, behaviour, sense of self, and language. It has been estimated that more than 21 million people are affected by schizophrenia worldwide, and it is associated with considerable disability. Many patients with schizophrenia experience hearing voices and delusions. Patients suffering from this condition are 2 to 2.5 times more prone to die prematurely (WHO (2016) Schizophrenia Fact Sheet). The neurophysiological basis of schizophrenia remains poorly understood, but many studies suggested that a dysregulation of cortical GABA transmission might be the cause of schizophrenia.

In a recent study, a gain-of-function missense variant in SLC12A2, encoding the bumetanide-sensitive NKCC1 co-transporter was identified in human schizophrenia. Functional experiments showed that this variant of NKCC1 is a gain-of-function variant, increasing Cl⁻ ion-dependent activity even in conditions in which the transporter is normally functionally silent (Merner, N. D., et al. 2016). Another study found a KCC loss-of-function variant in human schizophrenia (Merner, N. D., et al. 2015). In both the studies (gain-of-function of NKCC or loss-of-function of KCC) the Cl⁻ ion concentration is increased, which might lead to a disruption of GABA neurotransmission. The blocking of NKCC would lead to a normalization of Cl⁻ concentration and reverse the GABA signaling back to hyperpolarization.

Edema is another disorder that is a result of an imbalance of fluid between the capillary and intestinal spaces caused by venous obstruction, increased plasma volume, and increased capillary permeability that leads to swelling or puffiness. The imbalance of the extracellular fluid volume of the cell membrane is regulated by the kidneys by adjusting sodium and water excretion. The edema may be localized only in the limb or generalized and massive in the whole body. The treatment of edema consists of sodium restriction, diuretic use, and treatment of the underlying disorder. The drug bumetanide being a loop diuretic and the NKCC blocker prevents sodium reuptake in the loop of Henle and leads to sodium and water excretion. There are various types of edema, including peripheral edema, pulmonary edema, cerebral edema, macular edema, and lymphedema.

Cerebral edema, also known as brain edema, involves swelling in the brain and can be life-threatening. It can result from traumatic brain injury, stroke, brain tumors, infections, or certain metabolic disorders. The cerebral edema causes intracranial hypertension (ICH) which leads to severe outcomes for patients in the clinical setting. The treatment includes an effective anti-edema therapy that may significantly decrease mortality in a variety of neurological conditions. At present, drug treatment is a cornerstone in the management of cerebral edema and osmotherapy has been the mainstay of pharmacological therapy, wherein mannitol and hypertonic saline (HS) are the most commonly used osmotic agents. Further, the inhibitors of Na/H exchanger and NKCC attenuate the brain edema formation through the inhibition of excessive transportation of ions and water from the blood into the cerebral tissue (Deng Y., et al. 2016). Therefore, medications designed to inhibit NKCC can be utilized as a therapeutic approach for treating cerebral edema.

Another disorder includes down syndrome, which is a genetic disorder caused by the presence of an extra (third) copy of genetic material at chromosome 21. It is the most frequent genetic cause that leads to intellectual disability, occurring in approximately 1 in every 1000 births worldwide, which means that each year around 3000 to 5000 children are born with this disorder ((WHO (2017), Genes and chromosomal diseases). The adults and children, who suffer from Down syndrome express lower than normal intelligence quotients, learning deficits, and memory impairment.

Down syndrome is the result of a process called nondisjunction, where the genetic material fails to separate resulting in an extra chromosome (trisomy 21). The altered GABAergic transmission is shown to contribute considerably to the learning and memory deficits in mouse models. A recent publication has shown that bumetanide was able to restore normal GABAergic transmission and reduce cognitive impairments (Deidda, G., et al. 2015). This study suggests that bumetanide and its derivatives show a promising approach as a potential treatment for cognitive impairments in individuals with Down syndrome.

Another disorder includes hyponatremia, which is a condition characterized by low sodium levels in the blood and is a common electrolyte abnormality in hospitalized patients. When sodium levels become too low, water enters the cells and causes them to swell, which can lead to various symptoms ranging from mild to severe, depending on the level of sodium depletion and the speed at which it occurs. Chronic hyponatremia refers to a condition where there is a prolonged or on-going decrease in serum sodium levels (serum sodium concentration <135 mmol/l) in the blood over a period of time. Hyponatremia is associated with an increased level of morbidity and mortality and lacks an effective and well-tolerated means for its prevention or treatment. Notably, hyponatremia is reported in >20% of patients with heart failure and predicts prolonged hospital stay and increased mortality. Hyponatremia is the prime indicator of the Syndrome of Inappropriate Antidiuretic hormone (SIADH), which is characterized by the excessive release of antidiuretic hormone (ADH or vasopressin) from the pituitary gland or another source. The causes of SIADHs include malignancy, notably small cell lung cancer (SCLC), in which the malignant cells manufacture and release Arginine Vasopressin (AVP). Other causes include pulmonary disease in which AVP may be released in response to aberrant signals from intra-thoracic receptors or CNS as well as psychiatric conditions or drugs used for their treatment, which impact the central signaling to activate AVP release. The treatment of hyponatremia includes the use of loop diuretics such as furosemide (Lasix), which impairs the renal concentrating and diluting system. Accordingly, loop diuretics with additional salt intake have been used alone or with fluid restriction to inhibit renal free water reabsorption in patients with hyponatremia (e.g., cancer, heart failure, or SIADH). Mechanistically, loop diuretics prevent elaboration of osmolality in the renal medulla that provides osmotic force for the reabsorption of electrolyte-free water from the collecting ducts and thereby act as a physiological antagonist to the renal actions of arginine vasopressin (AVP). However, the recent EFFUSE-FLUID Randomized Controlled Clinical Trial (RCCT) in patients with SIADH failed to demonstrate effective treatment of hyponatremia in those randomized to fluid retention plus furosemide (20 or 40 mg once daily) plus 3 gm salt daily compared to fluid restriction alone. This may be a consequence of the failure to use furosemide once a day to inhibit the ongoing effects of AVP on the renal tubules because of the short duration of action of furosemide of between 4-6 hours.

Another disorder includes sleep apnea which is a common condition characterized by pauses in breathing or shallow breaths during sleep that prevent the body from getting enough oxygen. The pauses can last from a few seconds to minutes and may occur multiple times per hour. There are two types of sleep apnea: (1) obstructive sleep apnea (OSA) and (2) central sleep apnea. OSA is a sleep disorder that involves a significant decrease in airflow in the presence of breathing effort. It is due to recurrent episodes of airway collapse during sleep accompanied by arterial oxyhemoglobin desaturation. It is distinguished from central sleep apnea by the fact that in central sleep apnea breathing efforts are not present. There are various symptoms of OSA categorized in night time symptoms and daytime symptoms, wherein the night-time symptoms include snoring, gasping, choking, nocturia, insomnia, and restlessness and daytime symptoms include non-restorative sleep, morning headache, dry throat, excessive daytime sleepiness, fatigue, tiredness, cognitive deficit, decreased vigilance, morning confusion, personality and mood changes, sexual dysfunction, gastro-esophageal reflux and hypertension. In OSA, there is an increase in pharyngeal airway resistance during sleep, particularly at night when muscle tone decreases and relaxation occurs. This increase in airway resistance contributes to the characteristic obstruction of the upper airway seen in OSA. There is normally a translocation of 300 mL of fluid from the lower limbs into the circulation at night with a consequent increase in fluid volume of the neck. The OSA disorder may be improved by a maneuver that reduces the pooling of fluid in the legs or reduces body fluid volume (e.g., a diuretic therapy in patients with CHF). However, no effective treatment of OSA is available that can prevent this condition.

Yet another disorder includes glaucoma, which is the second leading cause of blindness worldwide (WHO (2004)), particularly in older adults. Glaucoma is a group of eye conditions that can lead to damage to the optic nerve, which transmits visual information from the eye to the brain. This damage is often caused by increased pressure within the eye, known as intraocular pressure (IOP). There are several types of glaucoma, including, angle-closure glaucoma, normal-tension glaucoma, and secondary glaucoma, wherein open-angle glaucoma is characterized by a gradual increase in IOP due to the slow clogging of drainage canals in the eye, meaning the fluid in the eye is not able to drain, because the ion channels are blocked. This leads to an increased pressure in the eye and can cause blindness. It tends to develop slowly over time and may not cause symptoms until significant damage has occurred. Since it develops slowly over time, there are only a few symptoms, and the patients do not recognize the slow loss of eyesight at first. Angle-closure glaucoma is also caused by the same factors as open-angle glaucoma, however, the onset happens much faster and is accompanied by symptoms, such as headache, blurred vision, and eye pain. Therefore, with the increase in the age of the individual, the need for additional treatment is growing every year.

U.S. Patent Publication No. 2003/0152622 A1 discloses an erodible, gastric-retentive dosage form for delivering a diuretic agent and exemplifies furosemide as a diuretic. It comprises of a hydrophilic polymer matrix that swells in gastric fluid to ensure stomach retention and erodes gradually over time. The dosage form is designed to provide a controlled release of the diuretic agent, matching the in vivo release profile with the in vitro profile obtained from United States Pharmacopeia (USP) disintegration tests.

U.S. Patent Publication No. 2007/0196482A1 discloses a sustained-release oral dosage form of torsemide or its pharmaceutically acceptable salts. This formulation is designed to achieve a mean urinary excretion rate of at least 200 μg/hr for 4 to 20 hours after a single dose. The dosage form of torsemide includes gum-based gelling agents such as xanthan and locust bean gums, wherein the active pharmaceutical ingredient is present at a concentration ranging from 1 wt. % to 80 wt. %, or 5 wt. % to 50 wt. %. It further discloses at least one controlled-release excipient, which is a one matrix component and is selected from the group consisting of hydroxy propyl cellulose (HPC), hydroxypropyl methyl cellulose (HPMC), glyceryl behenate, and a polyethylene glycol glyceride.

Patent application CN104744355A discloses a method for preparing torsemide and its derivatives, wherein, R₁, and R₂ groups can each be independently selected from hydrogen, a saturated or unsaturated linear hydrocarbon group with C₁-C₁₀ carbon atoms; a saturated or unsaturated branched hydrocarbon group with C₁-C₁₀ carbon atoms, and a cycloalkyl group with C₃-C₆ carbon atoms; or NR₁R₂ is a five to seven-membered nitrogen heterocycle.

Patent application U.S. Pat. No. 4,244,950A discloses derivatives of 4-amino-3-sulfonamido-pyridine that have diuretic properties. These derivatives are represented by a general formula (I), wherein R₁ can be hydrogen, an alkyl, cycloalkyl, carbamoyl, or sulfonamide radical, and R₂ can be an alkyl, cycloalkyl, aryl group, or R₁ and R₂ together with the nitrogen atom can form a saturated heterocyclic radical; X represents alkoxy radical, a heterocyclic amino radical, or secondary/tertiary amino radicals with specified substituents. The invention further discloses pharmaceutically acceptable acid salts of these compounds.

B Masereel et al. in a non-patent literature article titled “Na⁺, 2Cl⁻, K⁺ cotransport system as a marker of antihypertensive activity of new torasemide derivatives” discloses the synthesis of a series of torsemide-related compounds and a loop diuretic. The study examines the diuretic potency and inhibitory effects of these compounds on the erythrocyte and renal medullary thick ascending limb vesicles.

Hence, there is a growing need for a potent drug or composition, in an appropriate dosing form and formulation that specifically targets ion transporters, ensuring their proper function and averting any malfunctioning that leads to disorders associated with abnormal ion transport.

### Objective of the Invention

An objective of the present invention is to provide a compound, a method for synthesizing the compound and its use, which reduces the incidence of malfunctioning of ion channels that cause channelopathies and enhances the smooth flow of various cations and anions including Na⁺, K⁺, and Cl⁻ across the cell membrane to treat various diseases/disorders/conditions associated with defective ion channels such as cystic fibrosis, hypertension, hyperkalemia and hypokalemia, heart failure, kidney disease, gout, anxiety autism spectrum, hyperhidrosis disorders, spinal cord injury, stroke, schizophrenia, edema, down syndrome and the like.

Another objective of the present invention is to provide a sulfonyl carboxamide compound and pharmaceutically acceptable salts thereof that aim to address diverse requirements essential for effective ion transports.

Yet another object of the present invention is to provide a pyridine-substituted sulfonyl carboxamide composition that overcomes the deficiencies or drawbacks of the known composition or compound by specifically targeting ion transporters, ensuring their proper function and averting any malfunctioning that leads to disorders associated with abnormal ion transport.

The foregoing and other objects, features, and advantages of the present invention will become readily apparent upon further review of the following summary and detailed description of the preferred embodiment.

### SUMMARY OF THE INVENTION

This section provides a general summary of the disclosure and is not meant to be a comprehensive disclosure of the full scope of all its features.

In an aspect, the present invention provides a sulfonyl carboxamide compound of Formula (I) or a pharmaceutically acceptable salts thereof,

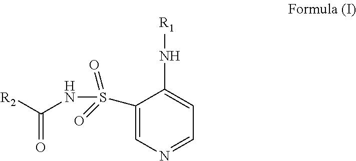

wherein R₁ is an aryl group, a cycloalkyl group of 3 to 6 carbon atoms, a heterocycloalkyl group of 3 to 5 carbon atoms, or a polycyclic group of 3 to 9 carbon atoms, and R₂ is an amine group, a bicyclic group of 5 or more carbon atoms.

In another embodiment, R₁ is selected from the groups represented by the formulas listed in Table 1.

TABLE 1

| | |
|---|---|
| 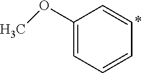 | (01) |
| 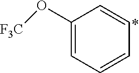 [SMILES: FC(F)(F)Oc1ccccc1] | (02) |
| 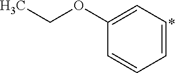 | (03) |
| 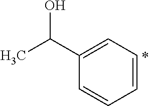 | (04) |
| 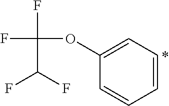 [SMILES: FC(F)C(F)(F)Oc1ccccc1] | (05) |
| 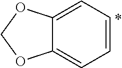 | (06) |
| 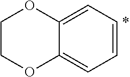 | (07) |
| 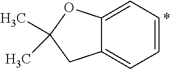 | (08) |
| 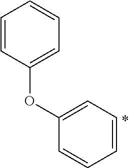 [SMILES: c1ccc(Oc2ccccc2)cc1] | (09) |
| 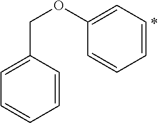 [SMILES: c1ccc(COc2ccccc2)cc1] | (10) |
| 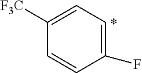 [SMILES: Fc1ccc(C(F)(F)F)cc1] | (11) |
| 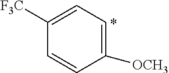 [SMILES: COc1ccc(C(F)(F)F)cc1] | (12) |
| 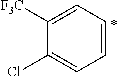 [SMILES: FC(F)(F)c1ccccc1Cl] | (13) |
| 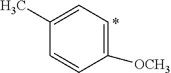 | (14) |
| 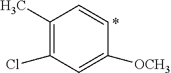 | (15) |
| 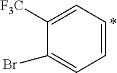 [SMILES: FC(F)(F)c1ccccc1Br] | (16) |
| 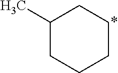 | (17) |
| 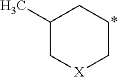 | (18) |
| 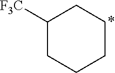 | (19) |
| 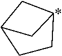 | (20) |
| 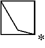 | (21) |
| 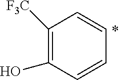 [SMILES: Oc1ccccc1C(F)(F)F] | (22) |
| 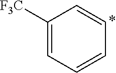 | (23) |
| 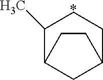 | (24) |
| 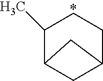 | (25) |
| 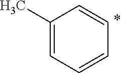 | (26) |

In the formulas involving R1 and R2 mentioned throughout the description, asterisks (*) indicate the positions where the R1 and R2 groups are attached to the compound of Formula I.

In another embodiment, R2 is a bicyclic derivative of sulfonyl carboxamide and is categorized into the following groups: spiro bicyclic derivatives, bicyclo derivatives, and aza-bicyclic derivatives which are represented by the formulas listed in Table 2.

TABLE 2

| | |
|---|---|
| 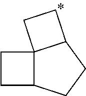 | (27) |
| 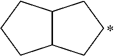 | (28) |
| 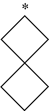 | (29) |
| 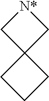 | (30) |
| 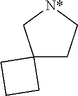 | (31) |
| 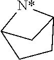 | (32) |
| 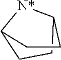 | (33) |
| 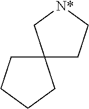 | (34) |
| 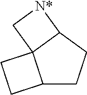 | (35) |
| 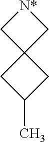 | (36) |
| 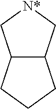 | (37) |
| 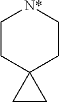 | (38) |
| 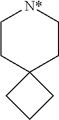 | (39) |
| 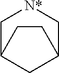 | (40) |
| 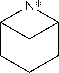 | (41) |
| 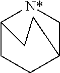 | (42) |
| 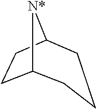 | (43) |
| 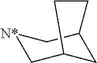 | (44) |
| 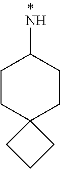 | (45) |
| 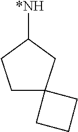 | (46) |
| 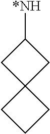 | (47) |
| 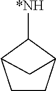 | (48) |
| 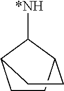 | (49) |
| 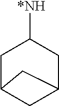 | (50) |
| 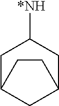 | (51) |
| 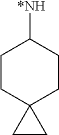 | (52) |
| 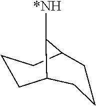 | (53) |
| 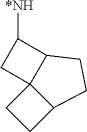 | (54) |

In a preferred embodiment, the R2 groups comprise aza-bicyclic derivatives, which are either aza-spiro derivatives or aza-bicyclo derivatives.

In another embodiment, the aza-bicyclic derivative comprises 6-12 atoms, where aza-bicyclo rings are either fused or bridged.

In another aspect, the present invention provides a method for preparing a sulfonyl carboxamide compound with a formula SAR001 or pharmaceutically acceptable salts thereof, comprising the steps of (i) reacting 4-nitro-2-(trifluoromethyl) phenol with K₂CO₃ and BnBr to obtain a compound with a formula 1-(Benzyloxy)-4-nitro-2-(trifluoromethyl)benzene; (ii) reacting the compound obtained at step (i) with SnCl2 to obtain a compound having the formula 4-(benzyloxy)-3-(trifluoromethyl) aniline; (iii) reacting the compound obtained at step (ii) with 4-Chloropyridine-3-sulfonamide in the presence of N,N-Diisopropylethylamine (DIPEA) to obtain a compound of formula 4-((4-(Benzyloxy)-3-(trifluoromethyl)phenyl)amino)pyridine-3-sulfonamide; (iv) reacting the compound obtained at step (iii) with isopropyl isocyanate to obtain a compound of formula 4-((4-(Benzyloxy)-3-(trifluoromethyl)phenyl)amino)-N (isopropylcarbamoyl)pyridine-3-sulfonamide; and (v) reacting the compound obtained at step (iv) with H2, Pd/C to obtain the resulting compound of formula represented by SAR001.

In an embodiment, the present invention provides a method for preparing a sulfonyl carboxamide compound with a formula of SAR002 or pharmaceutically acceptable salts thereof, comprising the steps of: (i) reacting 4-Chloropyridine-3-sulfonamide with 3-(trifluoromethyl) aniline in the presence of N,N-Diisopropylethylamine (DIPEA) to obtain a compound 4-((3-(Trifluoromethyl)phenyl)amino)pyridine-3-sulfonamide; and (ii) reacting the compound obtained at step (i) with isopropyl isocyanate to obtain the resulting compound having the formula represented by SAR002.

In another embodiment, the present invention provides a method for preparing a sulfonyl carboxamide compound with a formula of SAR003 or a pharmaceutically acceptable salts thereof, comprising the steps of (i) reacting 4-(m-tolylamino)pyridine-3-sulfonamide with ethyl chloroformate to obtain a compound Ethyl 4-(m-tolylamino)pyridin-3-ylsulfonylcarbamate; and (ii) reacting the compound obtained at step (i) with 2-azaspiro[3.3]heptane to obtain the resulting compound of N-(4-(m-Tolylamino)pyridin-3-ylsulfonyl)-2-azaspiro[3.3]heptane-2-carboxamide or the formula represented by SAR003.

In another embodiment, the present invention provides a method for preparing a sulfonyl carboxamide compound with a formula represented by SAR004 or a pharmaceutically acceptable salts thereof, comprising the steps of subjecting 4-(m-tolylamino)pyridine-3-sulfonamide to DMAP in ACN and diphenyl carbonate and further reacting with 6-Azaspiro[3.4]octane hydrogen chloride to obtain the resulting compound of the formula represented by SAR004.

In another embodiment, the present invention provides a method for preparing a sulfonyl carboxamide compound with a formula SAR005 or a pharmaceutically acceptable salts thereof, comprising the steps of subjecting 4-(m-tolylamino)pyridine-3-sulfonamide to DMAP in ACN and diphenyl carbonate and further reacting with 7-Azabicyclo[2.2.1]heptane hydrogen chloride to obtain the resulting compound represented by SAR005.

In another embodiment, the present invention provides a method for preparing a sulfonyl carboxamide compound with formula SAR006 and SAR007 or pharmaceutically acceptable salts thereof, comprising the steps of (i) reacting 4-(m-tolylamino)pyridine-3-sulfonamide with ethyl chloroformate to obtain a compound Ethyl 4-(m-tolylamino)pyridin-3-ylsulfonylcarbamate; and (ii) reacting the compound obtained at step (i) with piperidine and pyrrolidine to obtain the compounds of formulas represented by SAR006 and SAR007 respectively.

The invention in another aspect provides a pharmaceutical composition comprising a compound of formula (I), or pharmaceutically acceptable salts of Formula (I) in combination with one or more pharmaceutically acceptable excipients; and a method of preparing the composition comprising the compound of formula (I) or pharmaceutically acceptable salt thereof, and use of the same for the treatment of diseases related to ion transport across the cell membranes.

Other features, benefits, and advantages of the present invention will be apparent upon a review of the present disclosure, including the specification, abstract, and claims.

### DETAILED DESCRIPTION OF THE INVENTION

The embodiments herein and the various features and advantageous details thereof are explained more fully with reference to the non-limiting embodiments and the following description. Numerous variations, changes, and substitutions may occur to those skilled in the art without departing from the invention. It should be understood that various alternatives to the embodiments of the present disclosure herein may be employed.

At the outset, for ease of reference, certain terms used in this application and their meanings as used in this context are set forth. To the extent a term used herein is not defined below, it should be given the broadest definition persons in the pertinent art have given that term as reflected in at least one printed publication or issued patent. Further, the present techniques are not limited by the usage of the terms used in the application, as all equivalents, synonyms, new developments, and terms or techniques that serve the same or a similar purpose are considered to be within the scope of the present claims.

Below is a description of various embodiments of the invention. Before describing selected embodiments of the present disclosure in detail, it is to be understood that the present invention is not limited to any embodiment described herein. The disclosure and description herein are illustrative and explanatory of one or more presently preferred embodiments and variations thereof, and it will be appreciated by those skilled in the art that various changes in the structures, materials or characteristics which may be made without departing from the spirit of the invention. Those of ordinary skill in the art can readily use the present application as a basis for designing or modifying derivatives to achieve the same objectives and/or the same advantages as the embodiments herein. It is also to be understood by those of ordinary skill in the art that these equivalent examples do not depart from the spirit and scope of the present application. Although the methods disclosed herein have been described with reference to the specific operations that are carried out in a specific order, it should be understood that these operations can be combined, subdivided, or reordered to form an equivalent method without departing from the teachings of the present application.

The articles “a” and “an” as used herein mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

As used herein, the term “or” includes “and/or” and the term “and/or” includes any and all combinations of one or more of the associated listed items. Expressions such as “at least one of,” when preceding a list of elements, modify the entire list of elements and do not modify the individual elements of the list.

Embodiments of the present invention relate to a compound, a composition, a method of synthesis, and the use of composition or compound for the treatment of diseases related to ion transport (cations and anions) across the cell membranes. More specifically, the present invention provides a compound and a composition containing the compound, for reducing the incidence of malfunctioning of ion channels that cause channelopathies. Disclosed compounds enhance the smooth flow of various cations and anions including Na+, K+, and Cl− across the cell membrane to treat various diseases/disorders/conditions associated with defective ion channels such as cystic fibrosis, hypertension, hyperkalemia and hypokalemia, heart failure, kidney disease, gout, anxiety autism spectrum, hyperhidrosis disorders, spinal cord injury, stroke, schizophrenia, edema, down syndrome, and the like.

In an aspect, the present invention provides a sulfonyl carboxamide compound of Formula (I) or pharmaceutically acceptable salts thereof.

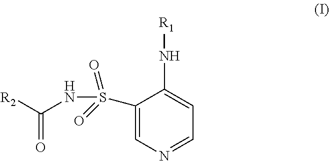

wherein R₁ is an aryl group, a cycloalkyl group of 3 to 6 carbon atoms, a heterocycloalkyl group of 3 to 5 carbon atoms, or a polycyclic group of 3 to 9 carbon atoms, and R₂ is an amine group, a bicyclic group of 5 or more carbon atoms.

The sulfonyl carboxamide moiety is a hybrid chemical structure derived from sulfonamides and carboxamides. Sulfonyl carboxamide motifs can be categorized into subtypes based on their pKa values and three-dimensional conformations.

The “alkyl group” of R1 includes linear or branched saturated monovalent hydrocarbon groups but is not limited to methyl group, ethyl group, propyl group, isobutyl group, sec-butyl group, tert-butyl group, pentyl group, iso-amyl group, and/or hexyl group.

In an embodiment, R1 may be selected from formulas (01) to (26). The “heteroatom (x)” of R1 having formulas (06), (07), (08), and (18) may refer to a group including 1 to 5 heteroatoms selected from N, O, S, Si, and P. The group containing heteroatoms may include multiple rings having 3-8 carbon atoms, wherein each ring of the multiple rings may be independently selected from at least aromatic, saturated, or partially unsaturated, and further may be fused, pendant, spirocyclic, or a combination thereof. The “aryl group” may comprise at least one aromatic ring and optionally a non-aromatic and/or carbocyclic ring. The “aryl groups” may be mono substituted, disubstituted, or trisubstituted selected from a methoxy, an ethoxy, a hydroxy, a trifluoromethyl, a trifluoromethoxy, a chlorine, a fluorine, a hydroxyethyl, a phenoxy, a tetrafluoroethoxy, and benzyloxy groups. The “aryl group” may refer to a monovalent functional group formed by the removal of one hydrogen atom from one or more rings of an arene, e.g., phenyl or naphthyl. The arene may refer to a hydrocarbon having an aromatic ring and includes monocyclic and/or polycyclic groups.

In an embodiment, R1 aryl group consists of the following formulas:

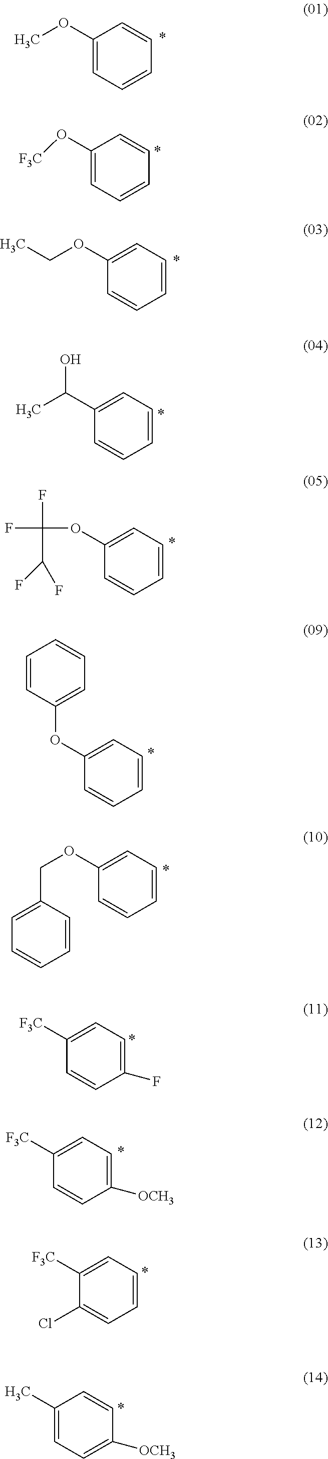

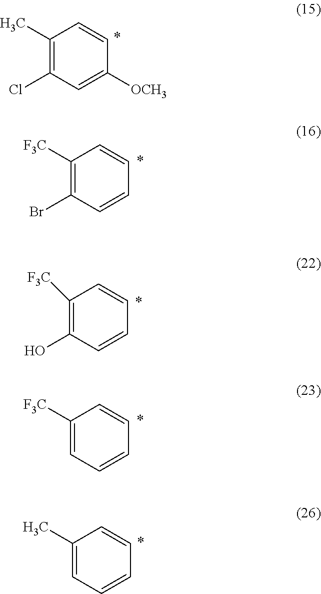

The “cycloalkyl group” may refer to a monovalent functional group having one or more saturated rings, wherein one or more saturated rings include carbon members e.g., a cyclopentyl group and a cyclohexyl group. In a preferred embodiment, the cycloalkyl groups may have 3-6 carbon atoms. The cycloalkyl groups may be monosubstituted, disubstituted, or trisubstituted with at least one trifluoromethyl group and one or more methyl groups.

In another embodiment, R1 cycloalkyl group is selected from the groups consists of the following formulas.

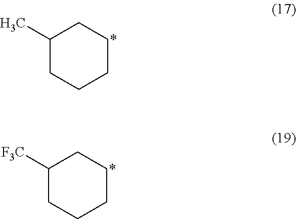

In another embodiment, R1 heterocycloalkyl group consists of the formula:

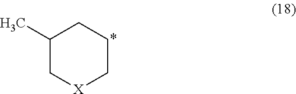

In another embodiment, R1 polycyclic group consists of the following formulas.

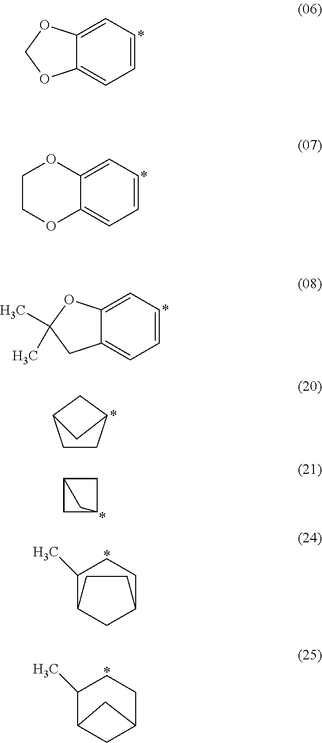

In another embodiment, R2 of Formula (I) is a bicyclic group of sulfonyl carboxamide and is categorized into the following groups: spiro bicyclic derivatives, bicyclo derivatives, and aza-bicyclic derivatives and are represented by the formulas listed in Table 2.

In an embodiment, the R2 group comprises spiro bicyclic derivatives represented by the following formulas:

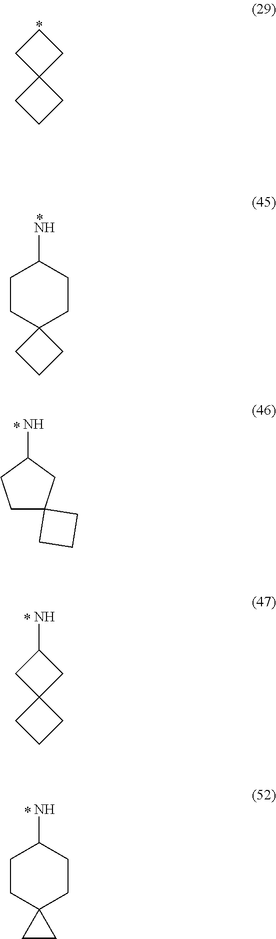 [SMILES: C1CC2(C1)CCC2 ; NC1CCC2(CCC2)CC1 ; NC1CCC2(CCC2)C1 ; NC1CC2(CCC2)C1 ; NC1CCC2(CC1)CC2]

In an embodiment, the R2 group comprises bicyclo derivatives represented by the following formulas:

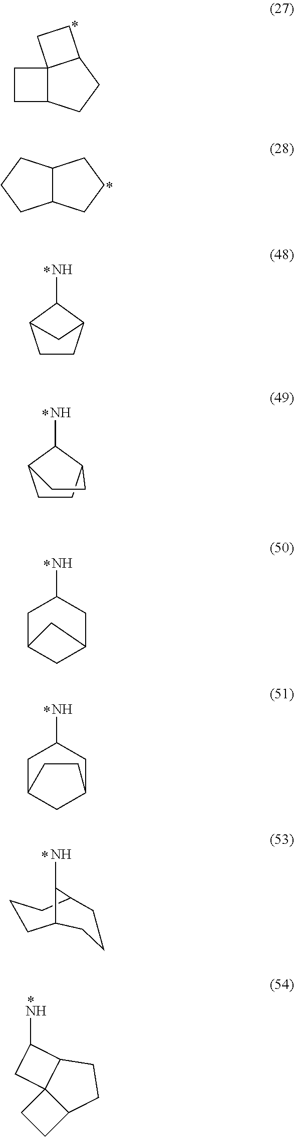

In a preferred embodiment, the R2 groups comprise aza-bicyclic derivatives, which are either aza-spiro derivatives or aza-bicyclo derivatives.

In another embodiment, the aza-bicyclic derivative comprises 6-12 atoms, where the aza-bicyclo rings are either fused or bridged.

In another embodiment, the R2 group comprises aza-spiro bicyclic derivatives of 6 to 9 atoms and may be selected from the following formulas:

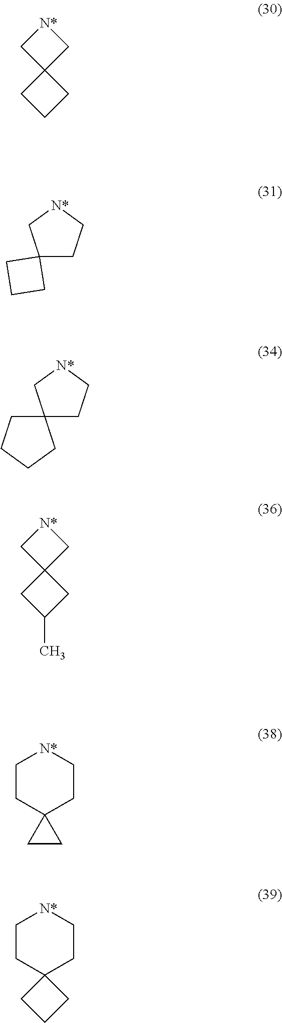

In another embodiment, the R2 group comprises aza-bicyclo derivatives of 6 to 9 atoms and may be selected from the following formulas:

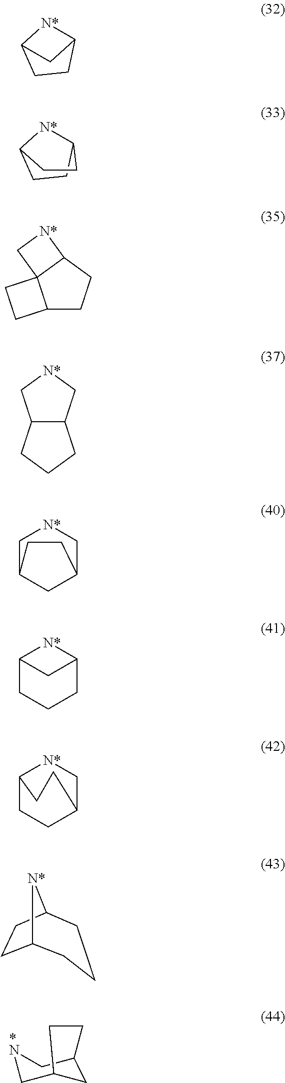

In an embodiment, the R2 group includes the amine group but is not limited to isopropylamine group, butylamine group, and pentylamine group.

Table 3 lists some of the compounds representing Formula (I) of the invention, though it is not limited to these compounds.

TABLE 3

| | Compound of formula (I): 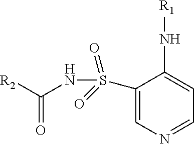 | |
|---|---|---|
| Compound name | R₁ | R₂ |
| SAR001 | 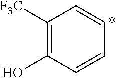 | 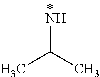 |
| SAR002 | 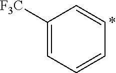 | 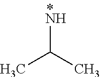 |
| SAR003 | 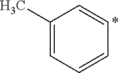 | 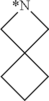 |
| SAR004 | 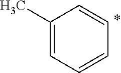 | 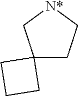 |
| SAR005 | 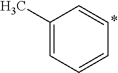 | 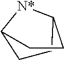 |
| SAR006 | 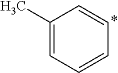 | 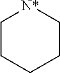 |
| SAR007 | 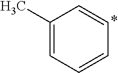 | 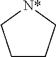 |
| SAR008 | 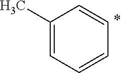 | 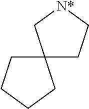 |
| SAR009 | 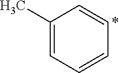 | 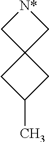 |
| SAR010 | 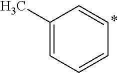 | 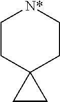 |
| SAR011 | 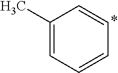 | 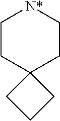 |
| SAR012 | 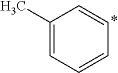 | 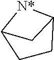 |
| SAR013 | 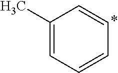 | 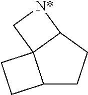 |
| SAR014 | 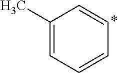 | 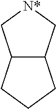 |
| SAR015 | 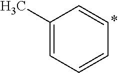 | 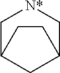 |
| SAR016 | 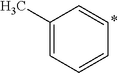 | 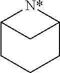 |
| SAR017 | 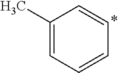 | 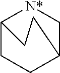 |
| SAR018 | 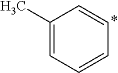 | 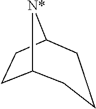 |
| SAR019 | 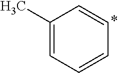 | 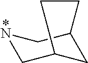 |

The present invention will be further described in detail with reference to the following examples, but it is not limited thereto. The following examples illustrate the preparation of compounds of Formula (I).

### Example 1

### Preparation of Compound SAR001

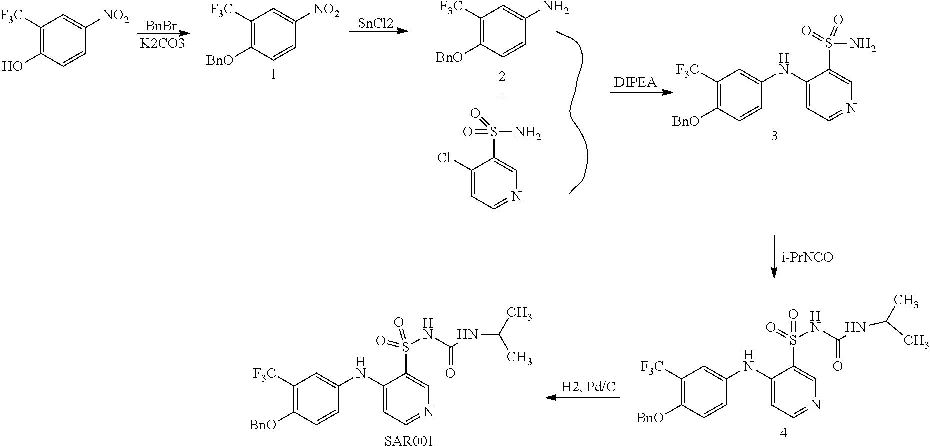

### Step 1: Preparation of compound 1-(Benzyloxy)-4-nitro-2-(trifluoromethyl)benzene (1)

K₂CO₃ (13.3 g, 96.6 mmol) was added to a solution of 4-nitro-2-(trifluoromethyl) phenol (10.0 g, 48.3 mmol) and BnBr (12.4 g, 72.5 mmol) in DMF (100 mL). The resulting mixture was heated at a temperature of 60° C. and stirred for 3 h. After cooling at room temperature, the mixture was filtered. The filtrate was diluted with water (300 mL) and then extracted with EtOAc (300 mL×3). The combined organic phases were washed with brine (500 mL×2), dried over anhydrous Na₂SO₄, and concentrated. The residue was purified by silica gel column chromatography (EtOAc/Hexane=3:1) to obtain the compound 1-(benzyloxy)-4-nitro-2-(trifluoromethyl)benzene (1) (13.2 g, 92% yield).

### Step 2: Preparation of compound 4-(Benzyloxy)-3-(trifluoromethyl) aniline (2)

SnCl₂·2H₂O (38.0 g, 168.4 mmol) was added to a solution of the obtained compound (1) (10.0 g, 33.7 mmol) in EtOH (150 mL). The resulting mixture was heated at the temperature of 70° C. and stirred for 2.5 h. After cooling at room temperature, the mixture was diluted with H₂O (300 mL) and then alkalified with 2N aqueous NaOH to pH=7.5˜8. The mixture was filtered and the filtrate was extracted with EtOAc (300 mL×3). The combined organic phases were washed with brine (500 mL×2), dried over anhydrous Na₂SO₄, and concentrated. The residue was purified by silica gel column chromatography (EtOAc/Hexane=1:2) to obtain the compound 4-(benzyloxy)-3-(trifluoromethyl) aniline (2) (8.1 g, 89% yield). LC-MS: m/z 268.2 [M+H]+.

### Step 3: Preparation of compound 4-((4-(Benzyloxy)-3-(trifluoromethyl)phenyl)amino)pyridine-3-sulfonamide (3)

4-Chloropyridine-3-sulfonamide (5.8 g, 30.0 mmol) was added to a solution of the obtained compound (2) (8.0 g, 30.0 mmol) in N-methylpyrrolidone (40 mL) at room temperature. The resulting mixture was heated at the temperature of 135° C. and stirred for 2 h. The reaction mixture was then cooled at room temperature, diluted with saturated aqueous sodium bicarbonate (200 mL), and then extracted with EtOAc (200 mL×3). The combined organic phases were washed with brine (300 mL×1), dried over anhydrous Na₂SO₄, and concentrated. The residue was purified by silica gel column chromatography (DCM/MeOH=20:1 to 10:1) to obtain the compound 4-((4-(benzyloxy)-3-(trifluoromethyl)phenyl)amino)pyridine-3-sulfonamide (3) (10.9 g, 86% yield). 1H-NMR (400 MHz, DMSO-d₆): δ 8.64 (s, 1H), 8.25 (d, J=5.6 Hz, 1H), 8.03 (s, 1H), 7.69 (br s, 2H), 7.50˜7.65 (m, 2H), 7.30˜7.50 (m, 6H), 6.80 (d, J=6.0 Hz, 1H), 5.31 (s, 2H).

### Step 4: Preparation of compound 4-((4-(Benzyloxy)-3-(trifluoromethyl)phenyl)amino)-N-(isopropylcarbamoyl) pyri dine-3-sulfonamide (4)

K₂CO₃ (2.35 g, 17 mmol) was added to a solution of the obtained compound (3) (5.5 g, 13.0 mmol) in N-methylpyrrolidone (50 mL) and water (5 mL). The mixture was heated at a temperature of 70° C. and stirred for 30 min. Then isopropyl isocyanate (1.2 g, 14.3 mmol) was added dropwise over 30 min. After completion of the addition, the reaction mixture was stirred at the temperature of 70° C. for an additional hour and then cooled at room temperature. The mixture was diluted with water (150 mL), acidified with acetic acid to pH=5˜6, and then extracted with EtOAc (200 mL×3). The combined organic phases were washed with brine solution (300 mL), dried over anhydrous Na₂SO₄, and concentrated to dryness. The residue was purified by silica gel column chromatography (DCM/MeOH=10:1) to obtain the compound 4-((4-(benzyloxy)-3-(trifluoromethyl)phenyl)amino)-N-(isopropylcarbamoyl) pyridine-3-sulfonamide (4) (5.4 g, 82% yield). LC-MS: m/z 509.6 [M+H]+.

### Step 5: Preparation of Compound SAR001

Pd/C (1 g) was added to a solution of the obtained compound (4) (5.4 g, 10.6 mmol) in MeOH (150 mL). The resulting mixture was stirred for 6 h at room temperature under a hydrogen atmosphere. Pd/C was filtered and the filtrate was concentrated to give the crude solid. The crude solid was triturated with DCM (100 mL), collected by filtration, and dried to obtain the compound SAR001 (3.7 g, 83% yield). 1H-NMR (400 MHz, DMSO-d6): δ 11.18 (br s, 1H), 10.81 (s, 1H), 8.95 (br s, 1H), 8.63 (s, 1H), 8.20 (d, J=6.4 Hz, 1H), 7.42 (dd, J=2.4, 8.4 Hz, 1H), 7.37 (d, J=2.4 Hz, 1H), 7.13 (d, J=8.4 Hz, 1H), 6.75 (d, J=6.4 Hz, 1H), 6.62 (br s, 1H), 3.64 (q, J=6.8 Hz, 1H), 1.02 (d, J=6.8 Hz, 6H). LC-MS: m/z 419.2 [M+H]⁺.

### Example 2

### Preparation of Compound SAR002

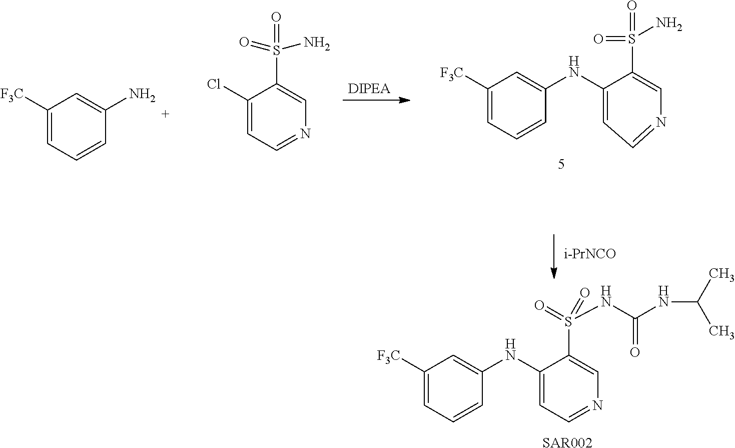

### Step 1: Preparation of compound 4-((3-(Trifluoromethyl)phenyl)amino)pyridine-3-sulfonamide (5)

4-Chloropyridine-3-sulfonamide (4.0 g, 20.8 mmol) was added to a solution of 3-(trifluoromethyl) aniline (6.7 g, 41.7 mmol) in N-methylpyrrolidone (40 mL) at room temperature. The resulting mixture was heated at the temperature of 135° C. and stirred for 2 h. The reaction mixture was cooled at room temperature and diluted with hexane (200 mL) and EtOAc (100 mL). The mixture was stirred for 0.5 h and the precipitate was collected by filtration. The solid was suspended in saturated aqueous sodium bicarbonate (200 mL) and then extracted with EtOAc (200 mL×3). The combined organic phases were washed with brine (200 mL×2), dried over anhydrous Na₂SO₄, and concentrated. The residue was purified by silica gel column chromatography (EtOAc/Hexane=1:2) to obtain the compound 4-((3-(trifluoromethyl)phenyl)amino)pyridine-3-sulfonamide (5) (5.4 g, 82% yield).

### Step 2: Preparation of Compound SAR002

K₂CO₃ (2.6 g, 18.5 mmol) was added to a solution of obtained compound (5) (4.9 g, 15.5 mmol) in N-methylpyrrolidone (60 mL) and water (3 mL). The mixture was heated at the temperature of 70° C. and stirred for 20 min. Then isopropyl isocyanate (1.5 g, 17.6 mmol) was added dropwise over 30 min. After completion of the addition, the reaction mixture was stirred at the temperature of 70° C. for an additional hour. After cooling at room temperature, the reaction mixture was diluted with water (200 mL), acidified with acetic acid to pH=5˜6, and then extracted with EtOAc (200 mL×3). The combined organic phases were washed with brine (300 mL), dried over anhydrous Na₂SO₄, and concentrated to dryness. The residue was purified by silica gel column chromatography (DCM/MeOH=20:1 to 10:1) to obtain the compound SAR002 (4.7 g, 75% yield). 1H-NMR (400 MHz, DMSO-d6): δ 11.11 (br s, 1H), 9.14 (br s, 1H), 8.72 (s, 1H), 8.33 (d, J=6.4 Hz, 1H), 7.55˜7.72 (m, 4H), 7.10 (d, J=6.4 Hz, 1H), 6.68 (br s, 1H), 3.64 (q, J=6.8 Hz, 1H), 1.02 (d, J=6.8 Hz, 6H). LC-MS: m/z 403.1 [M+H]+.

### Example 3

### Preparation of Compound SAR003

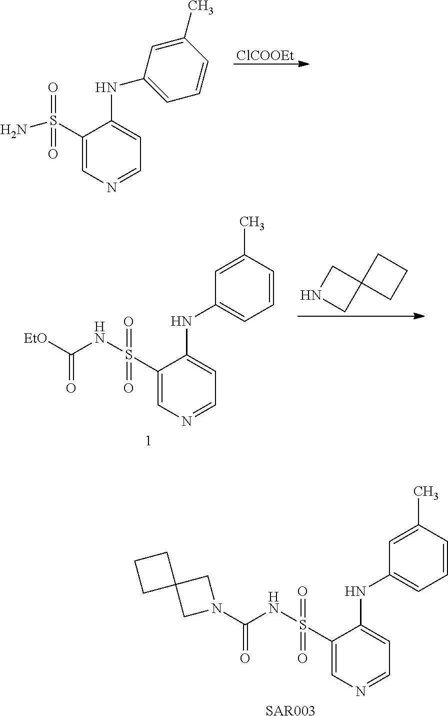

### Step 1: Preparation of compound Ethyl 4-(m-tolylamino)pyridin-3-ylsulfonylcarbamate (1)

NaOH (1.5 g, 38 mmol) was added to a suspension of 4-(m-tolylamino)pyridine-3-sulfonamide (10.0 g, 38 mmol) in acetone (200 mL) and H₂O (100 mL). The reaction mixture was stirred for 0.5 h at a temperature of 50° C. and then concentrated to dryness. The residue was suspended in acetone (100 mL), and ethyl chloroformate (11.2 g, 103 mmol) was added. The reaction mixture was stirred overnight at room temperature and then concentrated to obtain a crude compound (1) (25.5 g, ˜100% yield), which was used in the next step without further purification.

### Step 2: Preparation of compound N-(4-(m-Tolylamino)pyridin-3-ylsulfonyl)-2-azaspiro[3.3]heptane-2-carboxamide (SAR003)

A suspension of the obtained compound (1) (5.0 g, 15 mmol), 2-azaspiro[3.3]heptane oxalate (5.6 g, 30 mmol) and DIPEA (5.8 g, 45 mmol) in 1,4-dioxane (20 mL) was heated at a temperature of 80° C. and stirred for 12 h. The reaction mixture was cooled at room temperature, and the solvent was removed under reduced pressure. The residue was dissolved in EtOAc (200 mL) and washed with brine (50 mL×3), dried over anhydrous Na₂SO₄, concentrated, and then the residue was purified by column chromatography on silica gel (DCM/EtOAc/MeOH=10:10:1) to obtained SAR003 (1.1 g, 19% yield) as a yellow solid.

### Example 4

### Preparation of Compound SAR004

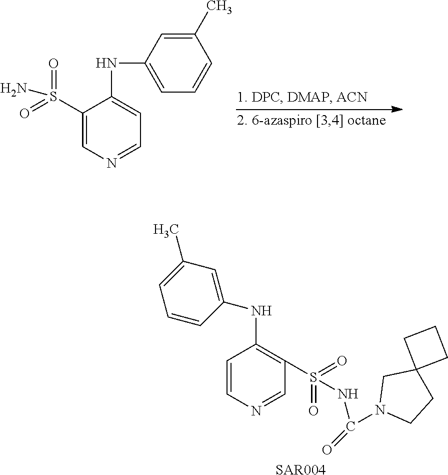 [SMILES: Cc1cccc(Nc2ccncc2S(N)(=O)=O)c1 ; Cc1cccc(Nc2ccncc2S(=O)(=O)NC(=O)N2CCC3(CCC3)C2)c1 ; "SAR004"]

To a solution of 4-(m-tolylamino)pyridine-3-sulfonamide (2.0 g, 7.6 mmol 1.0 eq), DMAP (1.86 g, 15.19 mmol 2.0 eq) in ACN (20 mL 10 V) was added diphenyl carbonate (DPC, 1.79 g, 8.36 mmol, 1.1 eq). The reaction mixture was stirred for 16 h until LCMS showed the starting sulfonamide was consumed. Then 6-Azaspiro[3.4]octane hydrogen chloride (845 mg, 7.61 mmol 1.0 eq) was added, and the reaction mixture was stirred at a temperature of 90° C. for 30 min. After cooling at room temperature, a citric acid solution (50 mL, IN) was added. The reaction mixture was extracted with EA (50 mL×2). The organic layers were combined, washed with brine (50 mL×2), dried over Na₂SO₄, filtered, and concentrated under a vacuum. The residue was purified by prep-HPLC (5% to 40%) to obtain the title compound SAR004 (1.05 g) as a white solid.

### Example 5

### Preparation of Compound SAR005

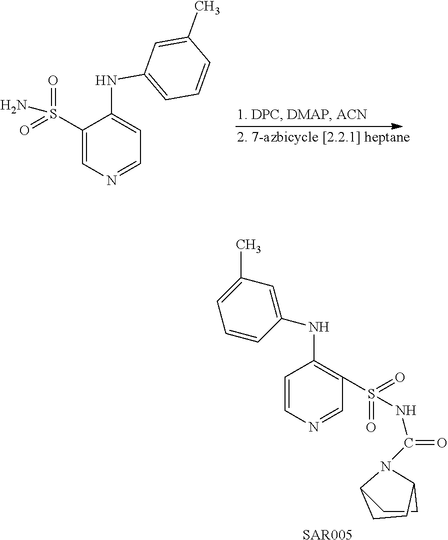 [SMILES: Cc1cccc(Nc2ccncc2S(N)(=O)=O)c1 ; Cc1cccc(Nc2ccncc2S(=O)(=O)NC(=O)N2C3CCC2CC3)c1 ; "SAR005"]

To a solution of 4-(m-tolylamino)pyridine-3-sulfonamide (3.2 g, 12.15 mmol 1.0 eq), DMAP (2.97 g, 24.3 mmol 2.0 eq) in ACN (30 mL 10 V) was added diphenyl carbonate (DPC, 2.86 g, 13.36 mmol, 1.1 eq). The reaction mixture was stirred for 16 h until LCMS showed the starting sulfonamide was consumed. Then 7-Azabicyclo[2.2.1]heptane hydrogen chloride (1.62 g, 12.15 mmol 1.0 eq) was added, and the reaction mixture was stirred at a temperature of 90° C. for 30 min. After cooling the reaction mixture at room temperature, a citric acid solution (100 mL, 1N) was added. The reaction mixture was extracted with ethyl acetate (100 mL×2) and the organic layers were combined, washed with brine (100 mL×2), dried over Na₂SO₄, filtered, and concentrated under a vacuum. The residue was purified by prep-HPLC (5%˜40%) to obtain the title compound SAR005 (1.2 g) as a white solid.

### Example 6

### Preparation of compound N-((4-(m-Tolylamino)pyridin-3-yl)sulfonyl)piperidine-1-carboxamide (SAR006)

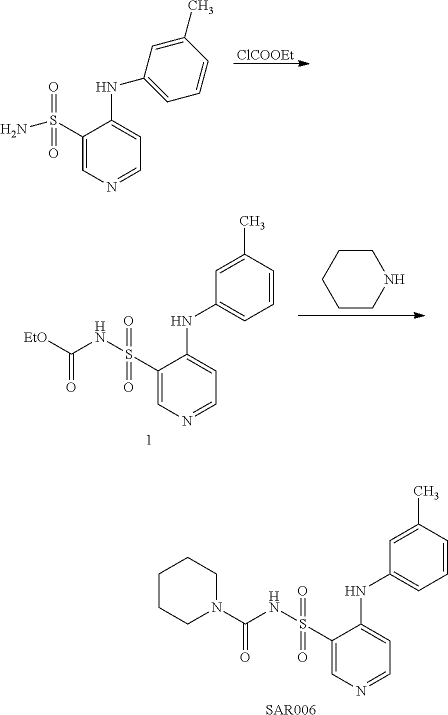

### Step 1: Preparation of compound Ethyl 4-(m-tolylamino)pyridin-3-ylsulfonylcarbamate (1)

NaOH (1.5 g, 38 mmol) was added to a suspension of 4-(m-tolylamino)pyridine-3-sulfonamide (10.0 g, 38 mmol) in acetone (200 mL) and H₂O (100 mL). The reaction mixture was stirred for 0.5 h at a temperature of 50° C., then concentrated to dryness. The residue was suspended in acetone (100 mL), and ethyl chloroformate (11.2 g, 103 mmol) was added. The reaction mixture was stirred overnight at room temperature and then concentrated to obtain crude compound (1) (25.5 g, ˜100% yield), which was used for the next step without further purification.

### Step 2: Preparation of compound N-((4-(m-Tolylamino)pyridin-3-yl)sulfonyl)piperidine-1-carboxamide (SAR006)

A mixture of the obtained compound (1) (10.0 g, 15 mmol), piperidine (5.1 g, 60 mmol), and NMP (50 ml) was heated at a temperature of 120° C. and stirred for 3 h. The reaction mixture was cooled at room temperature, and the solvent was removed under reduced pressure. The residue was dissolved in 0.5N aqueous NaOH (50 mL) and washed with EtOAc/hexane (v/v 1:3, 30 mL×3). The aqueous phase was acidified with IN HCl to a pH of 6˜7 and stirred for 0.5 h. The precipitated solid was collected by filtration, washed well with ACN, and then dried to obtain the compound SAR006 (4.1 g, 73% yield) as a yellow solid. ¹H-NMR (400 MHz, DMSO-d6): δ 12.94 (bs, 1H), 10.73 (bs, 1H), 8.61 (s, 1H), 8.17 (d, J=6.8 Hz, 1H), 7.39 (m, 1H), 7.00˜7.20 (m, 4H), 3.36 (m, 4H), 2.35 (s, 3H), 1.30˜1.60 (m, 6H). LC-MS: m/z 375.9 [M+H]⁺.

### Example 7

### Preparation of compound N-(4-(m-Tolylamino)pyridin-3-ylsulfonyl) pyrrolidine-1-carboxamide (SAR007)

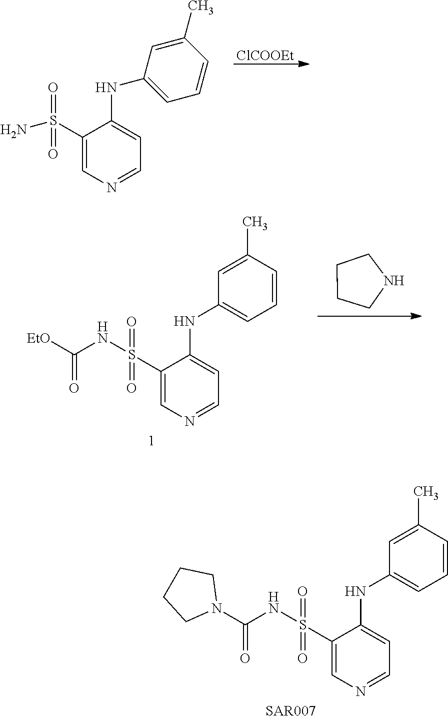

A mixture of compound Ethyl 4-(m-tolylamino)pyridin-3-ylsulfonylcarbamate (1) (10.0 g, 15 mmol), pyrrolidine (4.3 g, 60 mmol) and NMP (50 ml) was heated at a temperature of 120° C. and stirred for 3 h. The reaction mixture was cooled at room temperature, and the solvent was removed under reduced pressure. The residue was dissolved in 0.5N aqueous NaOH (50 mL) and washed with EtOAc/hexane (v/v 1:3, 30 mL×3). The aqueous phase was acidified with 1N HCl to a pH of 6˜7 and stirred for 0.5 h. The precipitated solid was collected by filtration, washed well with ACN, and then dried to obtain the compound SAR007 (3.2 g, 59% yield) as a yellow solid. ¹H-NMR (400 MHz, DMSO-d6): δ 12.20 (bs, 1H), 10.26 (bs, 1H), 8.63 (s, 1H), 8.18 (d, J=6.4 Hz, 1H), 7.38 (m, 1H), 7.00˜7.16 (m, 4H), 3.20˜3.30 (m, 4H), 2.35 (s, 3H), 1.60˜1.80 (m, 4H). LC-MS: m/z 361.6 [M+H]⁺.

The invention in another aspect provides a pharmaceutical composition comprising the compound of Formula (I), wherein R₂ groups are selected from aza-bicyclic derivatives, or pharmaceutically acceptable salts of Formula (I) in combination with one or more pharmaceutically-acceptable excipients.

In a preferred embodiment of the invention, a pharmaceutical composition comprising the compound of Formula (I), wherein R2 groups are selected from aza-spiro or aza-bicyclo derivatives, or pharmaceutically acceptable salts of Formula (I) in combination with one or more pharmaceutically-acceptable excipients is disclosed.

The pharmaceutical composition comprising the compound of formula (I) or a pharmaceutically acceptable salt thereof can be formulated in a solid dosage form for oral administration, and the solid dosage form can be powders, granules, capsules, tablets, or pills. In yet another embodiment, the solid dosage form can include one or more excipients such as fillers, binders, diluents, disintegrants, lubricants, glidants, coatings, solvents, solubilizers, preservatives, antioxidants, sweeteners, flavors, and buffers. The excipients used in the dosage forms are commonly known to a person of ordinary skill in the art.

The compound of formula (I) or a pharmaceutically acceptable salt thereof may be in the form of controlled-release formulation, or oral formulations for dosing patients. Dosing formulation depends on routes of drug administration that include topical (e.g., local or directly applied to an organ such as skin), enteral (e.g., system-wide administration often via gastrointestinal tract such as oral formulation of tablets, capsules, liquid syrup, etc.), and parenteral (systemic administration other than gastrointestinal tract such as epidural, intramuscular, intravenous, subcutaneous, nasal, sublingual, intradermal, intra-peritoneal, etc.). The oral formulations may include oral tablets, which can be formulated in an immediate-release or controlled-release form. Here the term controlled-release includes extended-release (ER), prolonged-release (PR), or sustained-release (SR) oral dosage forms. The controlled-release formulation comprises an effective amount of one or more active pharmaceutical ingredients (APIs) or compounds disclosed herein, and at least one controlled-release excipient. The controlled-release excipient can be classified as (1) reservoir systems including enteric-coated products; (2) osmotic systems; (3) ion-exchange resins; and (4) matrix systems. The matrix systems can further be subdivided into (a) monolithic matrix tablets; (b) erodible (hydrophobic) matrix tablets; and (c) gel-forming hydrophilic matrix tablets. Most monolithic matrix tablets use an inert matrix, which does not interact (inert) with biological fluids.

The main reason for the popularity of this system is drug release from the matrix is independent of the states and conditions of digestive juices, which shows quite large inter- and intra-patient variability. The matrix systems such as gel-forming hydrophilic or swellable matrix systems are homogeneous or heterogeneous systems in which the drug is dispersed in a swellable hydrophilic polymer. The drug release is a function of polymer characteristics. The most widely gel-forming polymer in the controlled-release segment is polyhydroxyethyl methacrylate (pHEMA). Because of their swelling capacity, several cellulose derivatives are applied as swelling gel-forming controlled release drug delivery excipients and most widely used is hydroxypropylmethyl-cellulose (HPMC). However, a variety of different molecular weight HPMCs are available and they vary in their release characteristics. Specifically, the viscosity and erosion/dissolution characteristic of the gel layer varies greatly and allows manipulations with expected drug released profile. Other swellable polymers used in matrix tablets are natural or artificial gum and dextrans. Erodible polymers such as polyanhydrides provide other types of excipients for controlled-release drugs with zero-order profiles.

In yet another embodiment of the present invention, a method for treatment of diseases related to ion transports is disclosed, said method of treatment includes administering to a patient in need of such treatment an effective amount of compound having Formula (I) or pharmaceutically acceptable salts thereof.

Various embodiments of the present invention provide advantages over the known compounds for the treatment of ion-transport-related diseases. Some potential advantages that are realizable by specifically targeting the ion transporters, to ensure their proper functioning and averting any malfunctioning that leads to disorders associated with abnormal ion transport.

Although the present invention has been illustrated and described herein with reference to preferred embodiments, it will be readily apparent to those of ordinary skill in the art that other embodiments may perform similar functions and/or achieve similar results. All such equivalent embodiments are within the spirit and scope of the present invention, are contemplated thereby, and are intended to be covered by the following claims.

The advantages set forth above, and those made apparent from the foregoing description, are efficiently attained. Since certain changes may be made in the above construction without departing from the scope of the invention, it is intended that all matters contained in the foregoing description as illustrative and not in a limiting sense. It is also to be understood that the following claims are intended to cover all the generic and specific features of the invention herein described, and all statements of the scope of the invention that, as a matter of language, might fall there within.

We claim:

1. A compound of formula (I): 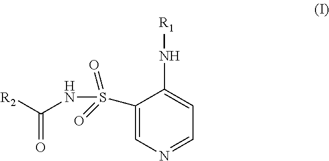
or a pharmaceutically acceptable salt thereof,
wherein R₁ is an aryl group, a cycloalkyl group of 3 to 6 carbon atoms, a heterocycloalkyl group of 3 to 5 carbon atoms, or polycyclic group of 3 to 9 carbon atoms, and
R₂ is a bicyclic group of 5 or more carbon atoms.

2. The compound of claim 1, wherein RI aryl group consists of the formulas:

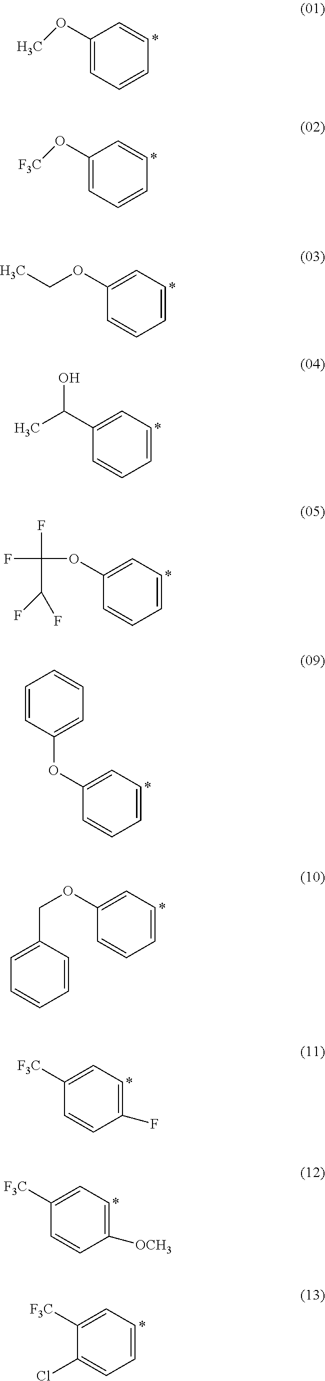

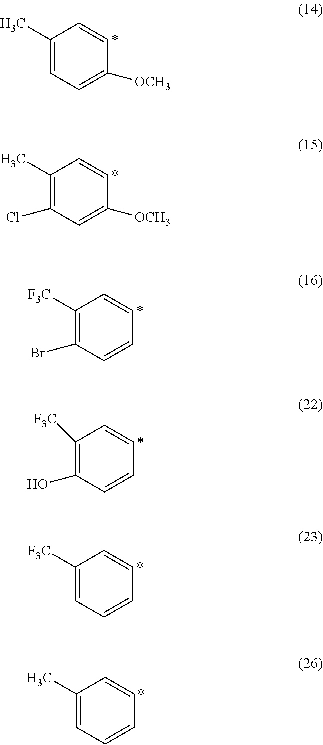

3. The compound of claim 1, wherein RI cycloalkyl group consists of the formulas.

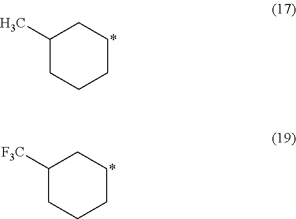

4. The compound of claim 1, wherein R₁ heterocycloalkyl group consists of the formula (18), wherein the heteroatom (x) is selected from O, N, S, Si, and P.

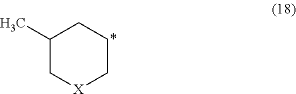

5. The compound of claim 1, wherein RI polycyclic group consists of the formulas.

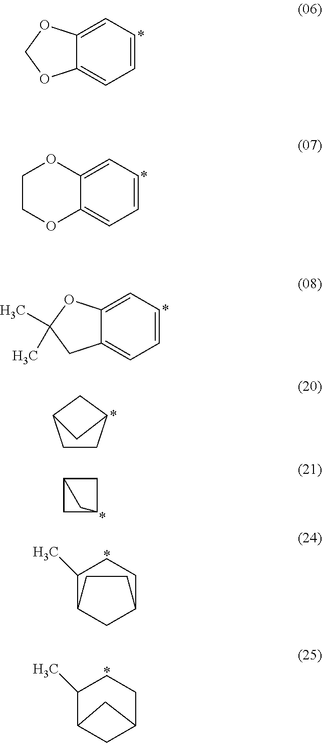

6. The compound of claim 1, wherein R₂ is bicyclic group comprises aza-bicyclic derivatives of 6-12 atoms.

7. The compound of claim 6, wherein the aza-bicyclic derivatives are either aza-spiro or aza-bicyclo derivatives of 6 to 9 atoms.

8. The compound of claim 7, wherein the aza-spiro derivatives are selected from formulas:

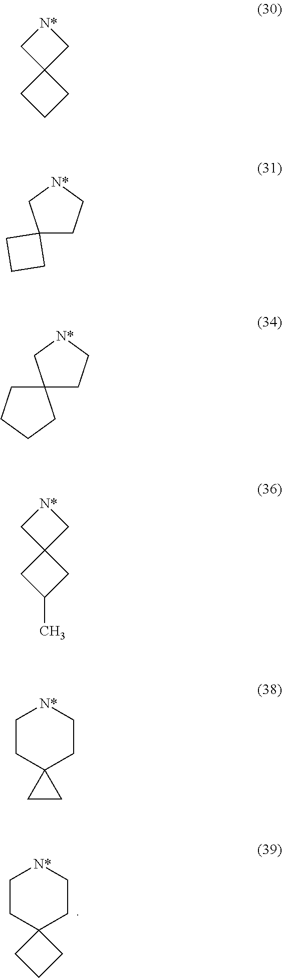

9. The compound of claim 7, wherein aza-bicyclo rings are either fused or bridged.

10. The compound of claim 9, wherein aza-bicyclo derivatives are selected from formulas:

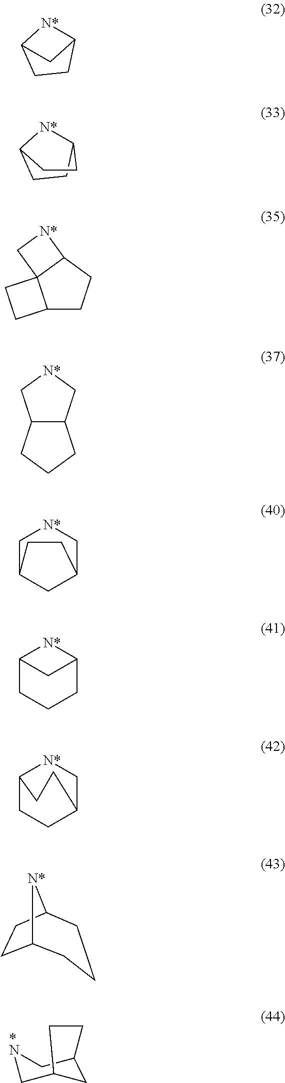

11. A pharmaceutical composition comprising the compound of formula (I), in combination with one or more pharmaceutically-acceptable excipients.

12. The pharmaceutical composition of claim 11, wherein the one or more pharmaceutically-acceptable excipients are controlled-release excipients.
